(19) United States
(12) Patent Application Publication
(10) Pub. No.: US 2013/0184555 A1
(43) Pub. Date: Jul. 18, 2013

(54) ORAL CAVITY MOUNTED PHOTOACOUSTIC SENSING UNIT

(71) Applicants: Bo Chen, Louisville, CO (US); Sarah Hayman, Boulder, CO (US); Youzhi Li, Longmont, CO (US); Friso Schlottau, Lyons, CO (US)

(72) Inventors: Bo Chen, Louisville, CO (US); Sarah Hayman, Boulder, CO (US); Youzhi Li, Longmont, CO (US); Friso Schlottau, Lyons, CO (US)

(73) Assignee: Nellcor Puritan Bennett LLC, Boulder, CO (US)

(21) Appl. No.: 13/348,865

(22) Filed: Jan. 12, 2012

(51) Int. Cl. A61B 6/00

(52) U.S. Cl. 600/407

(57) ABSTRACT

An oral cavity mounted photoacoustic sensing unit is disclosed. The sensing unit may include a light source, a photoacoustic detector, and a support assembly. The support assembly may be configured such that the light source and the photoacoustic detector are fixably located relative to the buccal or sublingual tissue of a subject.

The present disclosure relates to photoacoustic monitoring, and more particularly relates to an oral cavity mounted photoacoustic sensing unit.

### SUMMARY

A photoacoustic sensing unit may be configured to attach to the oral cavity of a subject for physiological monitoring.

In some embodiments, an oral cavity mounted photoacoustic sensing unit may include a light source, a photoacoustic sensing unit, and a support assembly. The support assembly may be configured such that the light source and the photoacoustic detector are fixably located relative to the buccal or sublingual tissue of a subject. The light source may illuminate a target area of the subject, resulting in an acoustic pressure response that may be detected by the photoacoustic detector. In some embodiments, the light source may include one or more fiber optic fibers. In some embodiments, the light source may include one or more optical elements (e.g., a prism or diffuser) configured to direct light to the target area of the subject. In some embodiments, multiple light sources may be used, where the light sources are arranged around the photoacoustic detector. In some embodiments, the photoacoustic detector may be a linear piezoelectric ultrasound receiver array or a multi-element detector array. In some embodiments, the photoacoustic detector may be a disk detector or an annular detector. In some embodiments, an acoustic transport medium may be used between the photoacoustic detector and the subject. The acoustic transport medium may be impedance matched to the photoacoustic response of the subject.

In some embodiments the oral cavity mounted photoacoustic sensor unit may include a processor coupled to the light source and the photoacoustic detector. The oral cavity mounted photoacoustic sensor unit may include a display for displaying information related to the operation of the photoacoustic sensor unit. For example, status information or a value of a physiological parameter may be displayed. The processor may be coupled to a transceiver to communicate with a physiological monitor over a wireless connection.

In some embodiments, the oral cavity mounted photoacoustic sensor unit may include an adjustable cuff to hold the light source and photoacoustic detector proximal to the buccal or sublingual tissue of the subject. In some embodiments, a pressure sensor may be used to detect the pressure of attachment of the oral cavity-mounted photoacoustic sensor unit to the subject. A power source may be coupled to the body mounted photoacoustic sensor unit to provide power, for example, to the light source, photoacoustic detector, processor, display and/or transceiver.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative physiological monitoring system in accordance with some embodiments of the present disclosure;

FIG. 2 is a block diagram of the illustrative physiological monitoring system of FIG. 1 coupled to a subject in accordance with some embodiments of the present disclosure;

FIG. 3 is a block diagram of an illustrative signal processing system in accordance with some embodiments of the present disclosure;

FIG. 4 is an illustrative photoacoustic arrangement in accordance with some embodiments of the present disclosure;

FIG. 5 is a plot of an illustrative photoacoustic signal, including peaks corresponding to walls of blood vessels in accordance with some embodiments of the present disclosure;

FIG. 6 is an illustrative perspective view of an oral cavity mounted photoacoustic sensing unit in accordance with some embodiments of the present disclosure;

FIG. 7 is a further illustrative perspective view of an oral cavity mounted photoacoustic sensing unit in accordance with some embodiments of the present disclosure;

FIG. 8 is an illustrative perspective view of an oral cavity mounted photoacoustic sensing unit mounted to the head of a subject in accordance with some embodiments of the present disclosure;

FIG. 9 is an illustrative perspective view of an oral cavity mounted photoacoustic sensing unit impinging on buccal tissue, in accordance with some embodiments of the present disclosure;

FIG. 10 is an illustrative plan view of an oral cavity mounted photoacoustic sensing unit support assembly in accordance with some embodiments of the present disclosure;

FIG. 11 is an illustrative plan view of an oral cavity mounted photoacoustic sensing head with an annular photoacoustic detector and a disk light source in accordance with some embodiments of the present disclosure;

FIG. 12 is an illustrative plan view of an oral cavity mounted photoacoustic sensing head with spot light sources and a disk detector in accordance with some embodiments of the present disclosure;

FIG. 13 is an illustrative plan view of an oral cavity mounted photoacoustic sensing head with spot light sources and array detector in accordance with some embodiments of the present disclosure;

FIG. 14 is an illustrative plan view of an oral cavity mounted photoacoustic sensing head with a diffused spot light source and array detector in accordance with some embodiments of the present disclosure;

FIG. 15 is an illustrative plan drawing of an optical element that may be employed in an oral cavity mounted photoacoustic sensing unit in accordance with some embodiments of the present disclosure; and

FIG. 16 is an illustrative plan drawing of a sublingual sensing unit in accordance with some embodiments of the present disclosure.

### DETAILED DESCRIPTION OF THE FIGURES

Photoacoustics (or “optoacoustics”) or “the photoacoustic effect” or (“the optoacoustic effect”) refers to the phenomenon in which one or more wavelengths of light are presented to and absorbed by one or more constituents of an object, thereby causing an increase in kinetic energy of the one or more constituents, which causes an associated pressure response within the object. Particular modulations or pulsing of the incident light, along with measurements of the corresponding pressure response in, for example, tissue of the subject, may be used for physiological parameter determination, medical imaging, or both. For example, oxygen saturation and/or the concentration of a constituent such as hemoglobin (oxygenated, deoxygenated, and/or total hemoglobin) may be determined using photoacoustic analysis.

A photoacoustic system may include a photoacoustic sensor that is placed at a site on a subject, typically a cheek, tongue, fingertip, toe, forehead or earlobe, or in the case of a neonate, across a foot. The photoacoustic system may use a light source, and any suitable light pipes (e.g., fiber optics), to pass light through the subject's tissue, or a combination of tissue thereof (e.g., organs) and an acoustic detector to sense the pressure response of the tissue induced by light absorption. Tissue may include muscle, fat, blood, blood vessels, and/or any other suitable tissue types. In some embodiments, the light source may be a laser or laser diode, operated in pulsed or continuous wave (CW) mode. In some embodiments, the acoustic detector may be an ultrasound detector, which may be suitable to detect pressure fluctuations arising from the constituent's absorption of to the incident light of the light source.

In some embodiments, the light from the light source may be focused, shaped, or otherwise spatially modulated to illuminate a particular region of interest. In some arrangements, photoacoustic monitoring may allow relatively higher spatial resolution than line of sight optical techniques (e.g., path integrated absorption measurements). The enhanced spatial resolution of the photoacoustic technique may allow for imaging, scalar field mapping, and other spatially resolved results, in 1, 2, or 3 spatial dimensions. The acoustic response to the photonic excitation may radiate from the illuminated region of interest, and accordingly may be detected at multiple positions.

The photoacoustic system may measure the pressure response that is received at the acoustic sensor as a function of time. The photoacoustic system may also include sensors at multiple locations. A signal representing pressure versus time or a mathematical manipulation of this signal (e.g., a scaled version thereof, etc.) may be referred to as the photoacoustic signal. The photoacoustic signal may be derived from a detected acoustic pressure signal by selecting a suitable subset of points of an acoustic pressure signal. The photoacoustic signal may be used to calculate any of a number of physiological parameters, including oxygen saturation and a concentration of a blood constituent (e.g., oxyhemoglobin), at a particular spatial location. In some embodiments, photoacoustic signals from multiple spatial locations may be used to construct an image (e.g., imaging blood vessels) or a scalar field (e.g., a hemoglobin concentration field). As used herein, blood vessels are understood to be the veins, arteries, and capillaries of a subject.

In some applications, the light passed through the tissue is selected to be of one or more wavelengths that are absorbed by the constituent in an amount representative of the amount of the constituent present in the tissue. The absorption of light passed through the tissue varies in accordance with the amount of the constituent in the tissue. For example, Red and/or infrared (IR) wavelengths may be used because highly oxygenated blood will absorb relatively less Red light and more IR light than blood with a lower oxygen saturation.

Any suitable light source may be used, and characteristics of the light provided by the light source may be controlled in any suitable manner. In some embodiments, a pulsed light source may be used to provide relatively short-duration pulses (e.g., nano-second pulses) of light to the region of interest. Accordingly, the use of a pulse light source may result in a relatively broadband acoustic response (e.g., depending on the pulse duration). The use of a pulsed light source will be referred to herein as the “Time Domain Photoacoustic” (TD-PA) technique. A convenient starting point for analyzing a TD-PA signal is given by Eq. 1:

p(z)=Γμₐφ(z) (1)

under conditions where the irradiation time is small compared to the characteristic thermal diffusion time determined by the properties of the specific tissue type. Referring to Eq. 1, p(z) is the photoacoustic signal (indicative of the maximum induced pressure rise, derived from an acoustic signal) at spatial location z indicative of acoustic pressure, Γ is the dimensionless Grüneisen parameter of the tissue, μₐ is the effective absorption coefficient of the tissue (or constituent thereof) to the incident light, and φ(z) is the optical fluence at spatial location z. The Grüneisen parameter is a dimensionless description of thermoelastic effects, and may be illustratively formulated by Eq. 2:

$$Γ = \frac{β {c}_{a}^{2}}{{C}_{P}} \qquad ( 2 )$$

where cₐ is the speed of sound in the tissue, β is the isobaric volume thermal expansion coefficient, and Cₚ is the specific heat at constant pressure. In some circumstances, the optical fluence, at spatial location z (within the subject's tissue) of interest may be dependent upon the light source, the location itself (e.g., the depth), and optical properties (e.g., scattering coefficient, absorption coefficient, or other properties) along the optical path. For example, Eq. 3 provides an illustrative expression for the attenuated optical fluence at a depth z:

φ(z)=φ₀e^{-μ}^{_{eff}}^{z} (3)

where φ₀ is the optical fluence from the light source incident at the tissue surface, z is the path length (i.e., the depth into the tissue in this example), and μ_{eff} is an effective attenuation coefficient of the tissue along the path length in the tissue in this example.

In some embodiments, a more detailed expression or model may be used rather than the illustrative expression of Eq. 3. In some embodiments, the actual pressure encountered by an acoustic detector may be proportional to Eq. 1, as the focal distance and solid angle (e.g., face area) of the detector may affect the actual measured photoacoustic signal. In some embodiments, an ultrasound detector positioned relatively farther away from the region of interest, will encounter a relatively smaller acoustic pressure. For example, the peak acoustic pressure signal received at a circular area A_{d} positioned at a distance R from the illuminated region of interest may be given by Eq. 4:

p_{d}=p(z)f(rₛ,R,A_{d}) (4)

where rₛ is the radius of the illuminated region of interest (and typically rₛ<R), and p(z) is given by Eq. 1. In some embodiments, the detected acoustic pressure amplitude may decrease as the distance R increases (e.g., for a spherical acoustic wave).

In some embodiments, a modulated CW light source may be used to provide a photonic excitation of a tissue constituent to cause a photoacoustic response in the tissue. The CW light source may be intensity modulated at one or more characteristic frequencies. The use of a CW light source, intensity modulated at one or more frequencies, will be referred to herein as the “Frequency Domain Photoacoustic” (FD-PA) technique. Although the FD-PA technique may include using frequency domain analysis, the technique may use time domain analysis, wavelet domain analysis, or any other suitable analysis, or any combination thereof. Accordingly, the term “frequency domain” as used in “FD-PA” refers to the frequency modulation of the photonic signal, and not to the type of analysis used to process the photoacoustic response.

Under some conditions, the acoustic pressure p(R,t) at detector position R at time t, may be shown illustratively by Eq. 5:

$$p ( R , t ) \sim \frac{{p}_{0} ( {r}_{0} , ω )}{R} {e}^{- iω ( t - τ )} \qquad ( 5 )$$

where r₀ is the position of the illuminated region of interest, ω is the angular frequency of the acoustic wave (caused by modulation of the photonic signal at frequency ω), R is the distance between the illuminated region of interest and the detector, and τ is the travel time delay of the wave equal to R/cₐ, where cₐ is the speed of sound in the tissue. The FD-PA spectrum p₀(r₀,ω) of acoustic waves is shown illustratively by Eq. 6:

$${p}_{0} ( {r}_{0} , ω ) = \frac{{Γμ}_{a} φ ( {r}_{0} )}{2 ( {μ}_{a} {c}_{a} - iω )} \qquad ( 6 )$$

where μₐcₐ represents a characteristic frequency (and corresponding time scale) of the tissue.

In some embodiments, a FD-PA system may temporally vary the characteristic modulation frequency of the CW light source, and accordingly the characteristic frequency of the associated acoustic response. For example, the FD-PA system may use linear frequency modulation (LFM), either increasing or decreasing with time, which is sometimes referred to as “chirp” signal modulation. Shown in Eq. 7 is an illustrative expression for a sinusoidal chirp signal r(t):

$$r ( t ) = cos ( ( {ω}_{0} + \frac{b}{2} t ) t ) \qquad ( 7 )$$

where ω₀ is a starting angular frequency, and b is the angular frequency scan rate. Any suitable range of frequencies (and corresponding angular frequencies) may be used for modulation such as, for example, 1-5 MHz, 200-800 kHz, or other suitable range, in accordance with the present disclosure. In some embodiments, signals having a characteristic frequency that changes as a nonlinear function of time may be used. Any suitable technique, or combination of techniques thereof, may be used to analyze a FD-PA signal. Two such exemplary techniques, a correlation technique and a heterodyne mixing technique, will be discussed below as illustrative examples.

In some embodiments, the correlation technique may be used to determine the travel time delay of the FD-PA signal. In some embodiments, a matched filtering technique may be used to process a photoacoustic signal. As shown in Eq. 8:

$${B}_{s} ( t - τ ) = \frac{1}{2 π} {\int }_{- ∞}^{∞} H ( ω ) S ( ω ) {e}^{iω t} d ω \qquad ( 8 )$$

Fourier transforms (and inverse transforms) are used to calculate the filter output Bₛ(t−T), in which H(ω) is the filter frequency response, S(ω) is the Fourier transform of the photoacoustic signal s(t), and T is the phase difference between the filter and signal. In some circumstances, the filter output of expression of Eq. 8 may be equivalent to an autocorrelation function. Shown in Eq. 9:

$$S ( ω ) = \frac{1}{2 π} {\int }_{- ∞}^{∞} s ( t ) {e}^{- iω t} d t \qquad ( 9 )$$

is an expression for computing the Fourier transform S(ω) of the photoacoustic signal s(t). Shown in Eq. 10:

H(ω)=S*(ω)e^{-iωτ} (10)

is an expression for computing the filter frequency response H(ω) based on the Fourier transform of the photoacoustic signal s(t), in which S*(ω) is the complex conjugate of S(ω). It can be observed that the filter frequency response of Eq. 10 requires the frequency character of the photoacoustic signal be known beforehand to determine the frequency response of the filter. In some embodiments, as shown by Eq. 11:

$$B ( t ) = {\int }_{- ∞}^{∞} r ( {t}^{′} ) s ( t + {t}^{′} ) d {t}^{′} \qquad ( 11 )$$

the known modulation signal r(t) may be used for generating a cross-correlation with the photoacoustic signal. The cross-correlation output B(t) of Eq. 11 is expected to exhibit a peak at a time t equal to the acoustic signal travel time τ. Assuming that the temperature response and resulting acoustic response follow the illumination modulation (e.g., are coherent), Eq. 11 may allow calculation of the time delay, depth information, or both.

In some embodiments, the heterodyne mixing technique may be used to determine the travel time delay of the FD-PA signal. The FD-PA signal, as described above, may have similar frequency character as the modulation signal (e.g., coherence), albeit shifted in time due to the travel time of the acoustic signal. For example, a chirp modulation signal, such as r(t) of Eq. 7, may be used to modulate a CW light source. Heterodyne mixing uses the trigonometric identity of the following Eq. 12:

cos(A)cos(B)=½[cos(A−B)−cos(A+B)] (12)

which shows that two signals may be combined by multiplication to give periodic signals at two distinct frequencies (i.e., the sum and the difference of the original frequencies). If the result is passed through a low-pass filter to remove the higher frequency term (i.e., the sum), the resulting filtered, frequency shifted signal may be analyzed. For example, Eq. 13 shows a heterodyne signal L(t):

$$L ( t ) = 〈 r ( t ) s ( t ) 〉 ≅ 〈 Kr ( t ) r ( t - \frac{R}{{c}_{a}} ) 〉 = \frac{1}{2} K cos ( \frac{R}{{c}_{a}} bt + θ ) \qquad ( 13 )$$

calculated by low-pass filtering (shown by angle brackets) the product of modulation signal r(t) and photoacoustic signal s(t). If the photoacoustic signal is assumed to be equivalent to the modulation signal, with a time lag R/cₐ due to travel time of the acoustic wave and amplitude scaling K, then a convenient approximation of Eq. 13 may be made, giving the rightmost expression of Eq. 13. Analysis of the rightmost expression of Eq. 13 may provide depth information, travel time, or both. For example, a fast Fourier transform (FFT) may be performed on the heterodyne signal, and the frequency associated with the highest peak may be considered equivalent to time lag Rb/cₐ. Assuming that the frequency scan rate b and the speed of sound cₐ are known, the depth R may be estimated.

Mucosa herein is understood to refer to a type of body tissue which lines a cavity or is otherwise exposed to the environment, and which includes primarily epithelial cells. For example, mucosae include the lining of the nostrils, mouth, esophagus, and stomach.

The oral mucosa lining the mouth is generally divided into the masticatory mucosa, the specialized mucosa, and the lining mucosa. Masticatory mucosa may include the gums and hard pallet. The specialized mucosa may include parts of the tongue. The lining mucosa may include the buccal mucosa and substantially all of the remaining lining of the oral cavity. The buccal mucosa may include the lining of the cheeks.

It is known that some of the properties of the tissues of the oral cavity, (e.g., sublingual, buccal) are closely correlated with other tissues of the gastrointestinal tract. For example, it has been shown that measurements of the dissolved blood carbon dioxide (P(CO₂)) content in the sublingual and gastric tissue of an animal model may be interchangeable. It has been shown that both buccal mucosal measurements and sublingual mucosal measurements may be reliable indicators of physiological conditions related to hemorrhagic shock. Given this, it will be understood that photoacoustic measurements of buccal, sublingual, other oral suitable tissues, or any combination thereof may provide information related to the gastric, esophageal, other suitable intestinal locations, or any combination thereof without performing more invasive measurements at these sites.

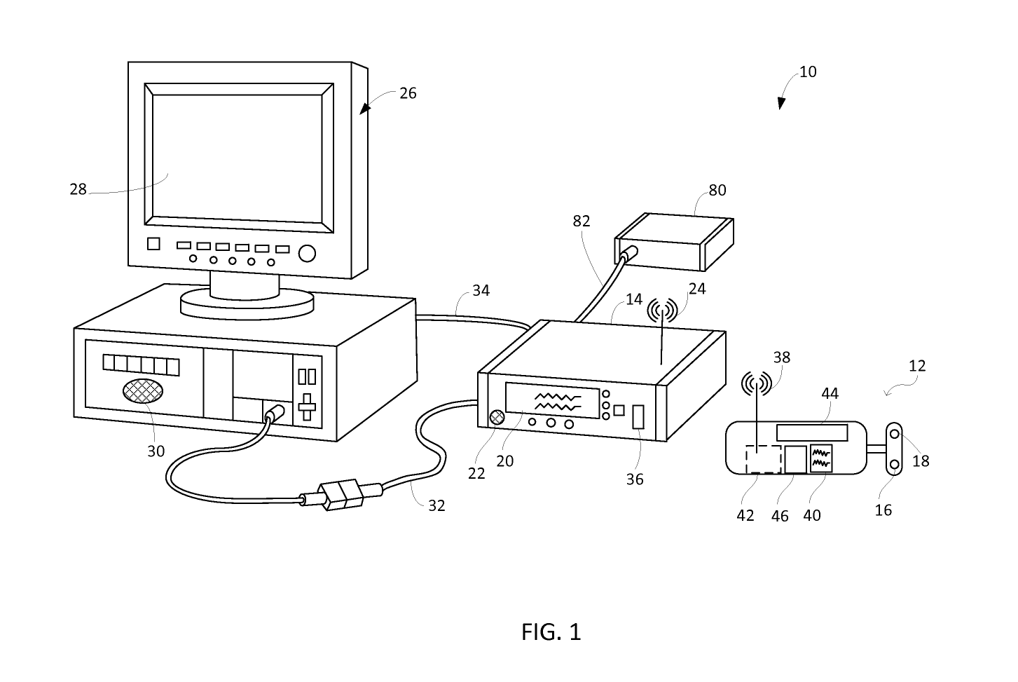

FIG. 1 shows an illustrative physiological monitoring system in accordance with some embodiments of the present disclosure. System 10 may include sensor unit 12 and monitor 14. In some embodiments, sensor unit 12 may be part of a photoacoustic monitor or imaging system. Sensor unit 12 may include a light source 16 for emitting light at one or more wavelengths into a subject's tissue, which may but need not correspond to visible light, into a subject's tissue. Light source 16 may provide a photonic signal including any suitable electromagnetic radiation such as, for example, a radio wave, a microwave wave, an infrared wave, a visible light wave, ultraviolet wave, any other suitable light wave, or any combination thereof. A detector 18 may also be provided in sensor unit 12 for detecting the acoustic (e.g., ultrasound) response that travels through the subject's tissue. Any suitable physical configuration of light source 16 and detector 18 may be used. In some embodiments, sensor unit 12 may include multiple light sources and/or acoustic detectors, which may be spaced apart. In an exemplary embodiment, sensor 12 may be an oral cavity mounted sensing unit and may include wireless transceiver 38, display 40, processing circuitry 42, and power source 44.

System 10 may also include one or more additional sensor units (not shown) that may take the form of any of the embodiments described herein with reference to sensor unit 12. An additional sensor unit may be the same type of sensor unit as sensor unit 12, or a different sensor unit type than sensor unit 12 (e.g., a photoplethysmograph sensor). Multiple sensor units may be capable of being positioned at two different locations on a subject's body; for example, a first sensor unit may be positioned on a subject's buccal tissue, while a second sensor unit may be positioned at a subject's fingertip, ankle, forehead, neck, or wrist.

In some embodiments, system 10 may include two or more sensors forming a sensor array in lieu of either or both of the sensor units. In some embodiments, a sensor array may include multiple light sources, detectors, or both. It will be understood that any type of sensor, including any type of physiological sensor, may be used in one or more sensor units in accordance with the systems and techniques disclosed herein. It will be understood that any number of sensors measuring any number of physiological signals may be used to determine physiological information in accordance with the techniques described herein.

In some embodiments, the sensor may be wirelessly connected to monitor 14 (e.g., via wireless transceivers 38 and 24) and include its own battery or similar power source 44. In some embodiments, sensor unit 12 may draw its power from monitor 14 and be communicate with monitor 14 via a physical connection such as a wired connection (not shown). Sensor unit 12, monitor 14, or both, may be configured to calculate physiological parameters based at least in part on data relating to light emission and acoustic detection received at one or more sensor units such as sensor unit 12. For example, sensor unit 12, monitor 14, or both, may be configured to determine blood oxygen saturation (e.g., arterial, venous, or both), hemoglobin concentration (e.g., oxygenated, deoxygenated, or total), pulse rate, blood pressure, any other suitable physiological parameters, or any combination thereof. In some embodiments, some or all calculations may be performed on sensor unit 12 (i.e., using processing circuitry 42) or an intermediate device and the result of the calculations may be passed to monitor 14. Further, monitor 14 may include a monitor display 20 configured to display the physiological parameters or other information about the system. Sensor unit 12 may also include a sensor display 40 configured to display the physiological parameters or other information about the system and a user interface 46. In an exemplary embodiment, processing circuitry 42 may be configured to operate light source 16 and detector 18 to generate and process photoacoustic signals, communicate with sensor display 40 to display values such as signal quality and power levels, receive signals from user input 46, and control wireless transceiver 38 to communicate data (e.g., photoacoustic output signals) with monitor 14.

In the embodiment shown, monitor 14 may also include a speaker 22 to provide an audible sound that may be used in various other embodiments, such as for example, sounding an audible alarm in the event that a subject's physiological parameters are not within a predefined normal range. In another embodiment, sensor unit 12 may communicate such information to the user, e.g., using sensor display 40, an audible source such as a speaker, vibration, tactile, or any other means for communicating a status to a user, such as for example, in the event that a subject's physiological parameters are not within a predefined normal range.

In some embodiments, sensor unit 12 may be communicatively coupled to monitor 14 via a wireless system, utilizing antenna 38 of sensor unit 12 and antenna 24 of monitor 14. Antenna 38 may be external or internal to sensor unit 12, and capable of transmitting signals, receiving signals, or both transmitting and receiving signals, via amplitude modulated RF, frequency modulated RF, Bluetooth, IEEE 802.11, WiFi, WiMax, cable, satellite, infrared, any other suitable transmission scheme, or any combination thereof. Communication between the sensor unit 12 and monitor 14 may also be carried over a cable (not shown) to an input 36 of monitor 14, or to a multi-parameter physiological monitor 26 (described below). The cable may include electronic conductors (e.g., wires for transmitting electronic signals from detector 18, or a partially or fully processed signal from sensor unit 12), optical fibers (e.g., multi-mode or single-mode fibers for transmitting emitted light from light source 16), any other suitable components, any suitable insulation or sheathing, or any combination thereof. Monitor 14 may include a sensor interface configured to receive physiological signals from sensor unit 12, provide signals and power to sensor unit 12, transfer data specific to the subject, general to the physiological parameter being measured, or both, or otherwise communicate with sensor unit 12. The sensor interface may include any suitable hardware, software, or both, which may allow communication between monitor 14 and sensor unit 12.

In some embodiments, sensor unit 12 may include an oral cavity mounted support assembly including light source 16 and detector 18, and a body mounted control assembly (not shown) including display 40, processing circuitry 42, user input 46, and wireless transceiver 38. Light source 16 may be located in the oral cavity mounted support assembly, the body mounted control assembly, or any combination thereof. For example, a laser diode light source may be located in the body mounted control assembly, coupled to the oral cavity mounted support assembly by a light pipe. As used herein, a light pipe may refer to a fiber optic cable, light guide, waveguide, prism, mirror, any other suitable arrangement for directing light from a first location to a second location, or any combination thereof.

In the illustrated embodiment, system 10 includes multi-parameter physiological monitor 26. The monitor 26 may include a cathode ray tube display, a flat panel display (as shown) such as a liquid crystal display (LCD) or a plasma display, or may include any other type of monitor now known or later developed. Multi-parameter physiological monitor 26 may be configured to calculate physiological parameters and to provide a monitor display 28 for information from sensor unit 12, monitor 14, or both, and from other medical monitoring devices or systems (not shown). For example, multi-parameter physiological monitor 26 may be configured to display an estimate of a subject's blood oxygen saturation, hemoglobin concentration, blood pressure, and/or pulse rate generated by sensor unit 12 or monitor 14. Multi-parameter physiological monitor 26 may include a speaker 30.

Monitor 14 may be communicatively coupled to multi-parameter physiological monitor 26 via a cable 32 or 34 that is coupled to a sensor input port or a digital communications port, respectively and/or may communicate wirelessly (not shown). The multi-parameter physiological monitor 26 may also be communicatively coupled to sensor unit 12 with or without the presence of monitor 14. Sensor unit 12 may be coupled to the multi-parameter physiological monitor 26 by a wireless connection using sensor wireless transceiver 38 and an antenna (not shown) on the multi-parameter physiological monitor, or by a cable (not shown). In addition, sensor unit 12, monitor 14, or monitor 26 may be coupled to a network to enable the sharing of information with servers or other workstations (not shown). In some embodiments this network may be a local area network, which may be further coupled through the internet or other wide area network for remote monitoring. Sensor unit 12, monitor 14 and multi-parameter physiological monitor 26 may be powered by a battery (not shown) or by a conventional power source such as a wall outlet.

Calibration device 80, which may be powered by monitor 14, a battery, or by a conventional power source such as a wall outlet, may include any suitable calibration device. Calibration device 80 may be communicatively coupled to monitor 14 via communicative coupling 82, and/or may communicate wirelessly (not shown). In some embodiments, calibration device 80 is completely integrated within monitor 14. In some embodiments, calibration device 80 may include a manual input device (not shown) used by an operator to manually input reference signal measurements obtained from some other source (e.g., an external invasive or non-invasive physiological measurement system).

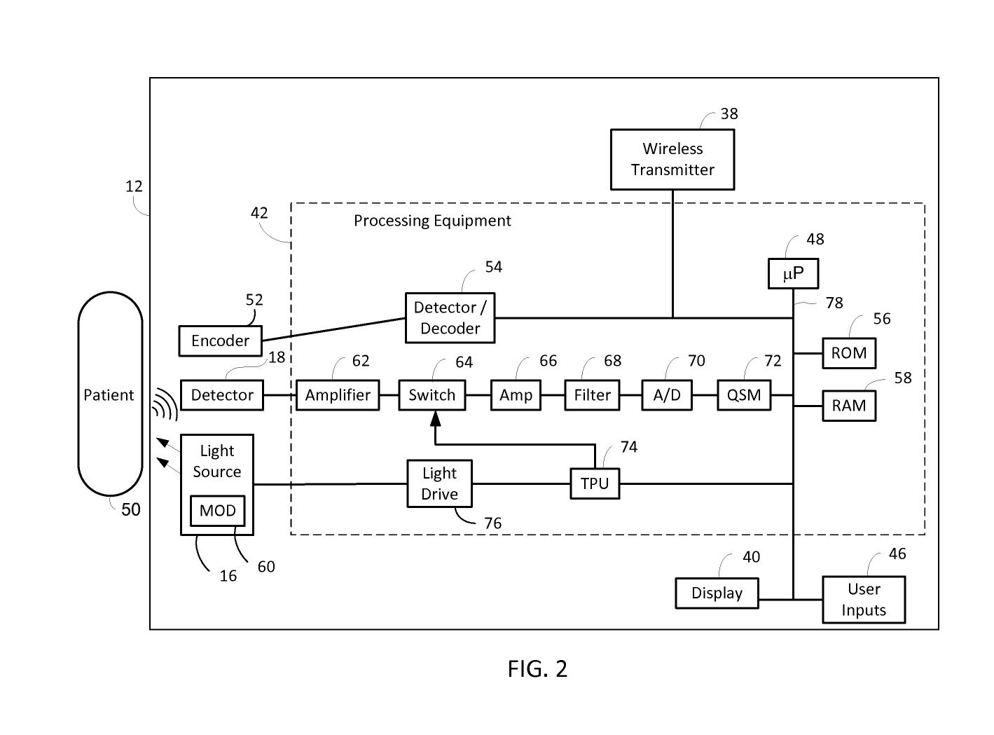

FIG. 2 is a block diagram of the illustrative physiological monitoring system of FIG. 1 coupled to a subject in accordance with some embodiments of the present disclosure. Physiological monitoring system 10 of FIG. 1 may be coupled to a subject's tissue 50 in accordance with an embodiment. Certain illustrative components of sensor unit 12 of FIG. 1 and monitor 14 of FIG. 1 are illustrated in FIG. 2. It will be understood that processing equipment 42 may be included fully or partially in monitor 14 of FIG. 1, fully or partially in sensor unit 12, fully or partially in multi-parameter physiological monitor 26, in any other suitable arrangement, or any combination thereof. It will be understood that any displayed information may be displayed on sensor display 40, monitor display 20, multi-parameter physiological monitor display 28, other suitable display, or any combination thereof.

Sensor unit 12 may include light source 16, detector 18, and encoder 52. In some embodiments, light source 16 may be configured to emit one or more wavelengths of light (e.g., visible, infrared) into a subject's tissue 50. Hence, light source 16 may provide Red light, IR light, any other suitable light, or any combination thereof, that may be used to calculate the subject's physiological parameters. In some embodiments, the Red wavelength may be between about 600 nm and about 700 nm, and the IR wavelength may be between about 800 nm and about 1000 nm. In embodiments where a sensor array is used in place of a single sensor, each sensor may be configured to provide light of a single wavelength. For example, a first sensor may emit only a Red light while a second may emit only an IR light. In a further example, the wavelengths of light used may be selected based on the specific location of the sensor.

It will be understood that, as used herein, the term “light” may refer to energy produced by electromagnetic radiation sources. Light may be of any suitable wavelength and intensity, and modulations thereof, in any suitable shape and direction. Detector 18 may be chosen to be specifically sensitive to the acoustic response of the subject's tissue arising from use of light source 16. It will also be understood that, as used herein, the “acoustic response” shall refer to pressure and changes thereof caused by a thermal response (e.g., expansion and contraction) of tissue to light absorption by the tissue or constituent thereof.

In some embodiments, detector 18 may be configured to detect the acoustic response of tissue to the photonic excitation caused by the light source. In some embodiments, detector 18 may be a piezoelectric transducer which may detect force and pressure and output an electrical signal via the piezoelectric effect. In some embodiments, detector 18 may be a Faby-Pérot interferometer, or etalon. For example, a thin film (e.g., composed of a polymer) may be irradiated with reference light, which may be internally reflected by the film. Pressure fluctuations may modulate the film thickness, thus causing changes in the reference light reflection which may be measured and correlated with the acoustic pressure. In some embodiments, detector 18 may be configured or otherwise tuned to detect acoustic response in a particular frequency range. Detector 18 may convert the acoustic pressure signal into an electrical signal (e.g., using a piezoelectric material, photodetector of a Faby-Pérot interferometer, or other suitable device). After converting the received acoustic pressure signal to an electrical, optical, and/or wireless signal, detector 18 may send the signal to processing equipment 42, where physiological parameters may be calculated based on the photoacoustic activity within the subject's tissue 50. The signal outputted from detector 18 and/or a pre-processed signal derived thereof, will be referred to herein as a photoacoustic signal.

In some embodiments, encoder 52 may contain information about sensor unit 12, such as what type of sensor it is (e.g., where the sensor is intended to be placed on a subject), the wavelength(s) of light emitted by light source 16, the intensity of light emitted by light source 16 (e.g., output wattage or Joules), the mode of light source 16 (e.g., pulsed versus CW), any other suitable information, or any combination thereof. This information may be used by processing equipment 42 to select appropriate algorithms, lookup tables and/or calibration coefficients stored in processing equipment 42 for calculating the subject's physiological parameters.

Encoder 52 may contain information specific to subject's tissue 50, such as, for example, the subject's age, weight, and diagnosis. This information about a subject's characteristics may allow processing equipment 42 to determine, for example, subject-specific threshold ranges in which the subject's physiological parameter measurements should fall and to enable or disable additional physiological parameter algorithms. Encoder 52 may, for instance, be a coded resistor that stores values corresponding to the type of sensor unit 12 or the type of each sensor in the sensor array, the wavelengths of light emitted by light source 16 on each sensor of the sensor array, and/or the subject's characteristics. In some embodiments, encoder 52 may include a memory on which one or more of the following information may be stored for communication to processing equipment 42: the type of the sensor unit 12; the wavelengths of light emitted by light source 16; the particular acoustic range that each sensor in the sensor array is monitoring; the particular acoustic spectral characteristics of a detector; a signal threshold for each sensor in the sensor array; any other suitable information; or any combination thereof.

In some embodiments, signals from detector 18 and encoder 52 may be transmitted to processing equipment 42. In the embodiment shown, processing equipment 42 may include a general-purpose microprocessor 48 connected to an internal bus 78. Microprocessor 48 may be adapted to execute software, which may include an operating system and one or more applications, as part of performing the functions described herein. Also connected to bus 78 may be a read-only memory (ROM) 56, a random access memory (RAM) 58, user inputs 46, sensor display 40, and a speaker (not shown).

RAM 58 and ROM 56 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium that can be used to store the desired information and that can be accessed by components of the system.

In the embodiment shown, a time processing unit (TPU) 74 may provide timing control signals to light drive circuitry 60, which may control the activation of light source 16. For example, TPU 74 may control pulse timing (e.g., pulse duration and inter-pulse interval) for TD-PA monitoring system. TPU 74 may also control the gating-in of signals from detector 18 through amplifier 62 and switching circuit 64. The received signal from detector 18 may be passed through amplifier 66, low pass filter 68, and analog-to-digital converter 70. The digital data may then be stored in a queued serial module (QSM) 72 (or buffer) for later downloading to RAM 58 as QSM 72 is filled. In some embodiments, there may be multiple separate parallel paths having components equivalent to amplifier 62, filter 68, and/or analog-to-digital converter 70 for multiple light wavelengths or spectra received. Any suitable combination of components (e.g., microprocessor 48, RAM 58, analog to digital converter 70, any other suitable component shown or not shown in FIG. 2) coupled by bus 78 or otherwise coupled (e.g., via an external bus), may be referred to as “processing equipment.”

In the embodiment shown, light source 16 may include modulator 60, in order to, for example, perform FD-PA analysis. Modulator 60 may be configured to provide intensity modulation, spatial modulation, any other suitable optical signal modulations, or any combination thereof. For example, light source 16 may be a CW light source, and modulator 60 may provide intensity modulation of the CW light source such as using a linear sweep modulation. In some embodiments, modulator 44 may be included in light drive 60, or other suitable components of physiological monitoring system 10, or any combination thereof.

In some embodiments, microprocessor 48 may determine the subject's physiological parameters, such as SpO₂, SvO₂, total hemoglobin concentration (t_{HB})_{,} oxyhemoglobin concentration, deoxyhemoglobin concentration, and/or pulse rate, using various algorithms and/or look-up tables based on the value of the received signals and/or data corresponding to the acoustic response received by detector 18. Signals corresponding to information about subject 50, and particularly about the acoustic signals emanating from a subject's tissue over time, may be transmitted from encoder 52 to decoder 54. These signals may include, for example, encoded information relating to subject characteristics. Decoder 54 may translate these signals to enable the microprocessor to determine the thresholds based at least in part on algorithms or look-up tables stored in ROM 56. In some embodiments, user inputs 46 may be used enter information, select one or more options, provide a response, input settings, any other suitable inputting function, or any combination thereof. User inputs 46 may be used to enter information about the subject, such as, for example, age, weight, height, diagnosis, medications, treatments, and so forth. In some embodiments, sensor display 40 may exhibit a list of values, which may generally apply to the subject, such as, for example, age ranges or medication families, which the user may select using user inputs 46.

The acoustic signal attenuated by the tissue of subject 50 can be degraded by noise, among other sources. Movement of the subject may also introduce noise and affect the signal. For example, the contact between the detector and the skin, or the light source and the skin, can be temporarily disrupted when movement causes either to move away from the skin. Another potential source of noise is electromagnetic coupling from other electronic instruments.

Noise (e.g., from subject movement) can degrade a sensor signal relied upon by a care provider, without the care provider's awareness. This is especially true if the monitoring of the subject is remote, the motion is too small to be observed, or the care provider is watching the instrument or other parts of the subject, and not the sensor site. Processing sensor signals may involve operations that reduce the amount of noise present in the signals, control the amount of noise present in the signal, or otherwise identify noise components in order to prevent them from affecting measurements of physiological parameters derived from the sensor signals.

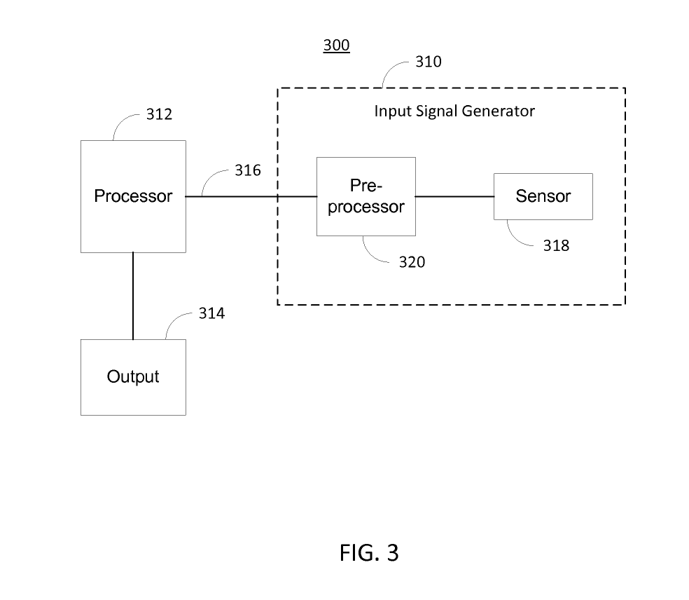

FIG. 3 is a block diagram of an illustrative signal processing system in accordance with some embodiments of the present disclosure. Signal processing system 300 may implement the signal processing techniques described herein. In some embodiments, signal processing system 300 may be included in a physiological monitoring system (e.g., physiological monitoring system 10 of FIGS. 1-2). In the illustrated embodiment, input signal generator 310 generates an input signal 316. As illustrated, input signal generator 310 may include pre-processor 320 coupled to sensor 318, which may provide input signal 316. In some embodiments, pre-processor 320 may be a photoacoustic module and input signal 316 may be a photoacoustic signal. In some embodiments, pre-processor 320 may be any suitable signal processing device and input signal 316 may include one or more photoacoustic signals and one or more other physiological signals, such as a photoplethysmograph signal. It will be understood that input signal generator 310 may include any suitable signal source, signal generating data, signal generating equipment, or any combination thereof to produce input signal 316. Input signal 316 may be a single signal, or may be multiple signals transmitted over a single pathway or multiple pathways.

Pre-processor 320 may apply one or more signal processing operations to the signal generated by sensor 318. For example, pre-processor 320 may apply a pre-determined set of processing operations to the signal provided by sensor 318 to produce input signal 316 that can be appropriately interpreted by processor 312, such as performing A/D conversion. In some embodiments, A/D conversion may be performed by processor 312. Pre-processor 320 may also perform any of the following operations on the signal provided by sensor 318: reshaping the signal for transmission, multiplexing the signal, modulating the signal onto carrier signals, compressing the signal, encoding the signal, and filtering the signal.

In some embodiments, input signal 316 may be coupled to processor 312. Processor 312 may be any suitable software, firmware, hardware, or combination thereof for processing input signal 316. For example, processor 312 may include one or more hardware processors (e.g., integrated circuits), one or more software modules, and computer-readable media such as memory, firmware, or any combination thereof. Processor 312 may, for example, be a computer or may be one or more chips (i.e., integrated circuits). Processor 312 may, for example, include an assembly of analog electronic components. Processor 312 may calculate physiological information. For example, processor 312 may perform time domain calculations, spectral domain calculations, time-spectral transformations (e.g., fast Fourier transforms, inverse fast Fourier transforms), any other suitable calculations, or any combination thereof. Processor 312 may perform any suitable signal processing of input signal 316 to filter input signal 316, such as any suitable band-pass filtering, adaptive filtering, closed-loop filtering, any other suitable filtering, and/or any combination thereof. Processor 312 may also receive input signals from additional sources (not shown). For example, processor 312 may receive an input signal containing information about treatments provided to the subject. Additional input signals may be used by processor 312 in any of the calculations or operations it performs in accordance with processing system 300.

In some embodiments, all or some of pre-processor 320, processor 312, or both, may be referred to collectively as processing equipment. For example, processing equipment may be configured to amplify, filter, sample and digitize input signal 316 (e.g., using an analog to digital converter), and calculate physiological information from the digitized signal.

Processor 312 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. In some embodiments, processor 312 may store physiological measurements or previously received data from signal 316 in a memory device for later retrieval. In some embodiments, processor 312 may store calculated values, such as blood oxygen saturation (e.g., arterial, venous, or both), hemoglobin concentration (e.g., oxygenated, deoxygenated, or total), pulse rate, blood pressure, or any other suitable calculated values, or combinations thereof, in a memory device for later retrieval.

Processor 312 may be coupled to output 314. Output 314 may be any suitable output device such as one or more medical devices (e.g., a medical monitor that displays various physiological parameters, a medical alarm, or any other suitable medical device that either displays physiological parameters or uses the output of processor 312 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 300 may be incorporated into system 10 (FIG. 1) in which, for example, input signal generator 310 may be implemented as part of sensor unit 12 (FIGS. 1 and 2) and monitor 14 (FIG. 1), processing equipment 42 (FIG. 2), and processor 312 may be implemented as part of monitor 14 (FIG. 1), and processing equipment 42 (FIG. 2). In some embodiments, portions of system 300 may be configured to be portable. For example, all or part of system 300 may be embedded in a small, compact object carried with or attached to the subject (e.g., a watch, other piece of jewelry, or a smart phone). In some embodiments, a wireless transceiver (not shown) may also be included in system 300 to enable wireless communication with other components of system 10 (FIG. 1). As such, system 10 (FIG. 1) may be part of a fully portable and continuous physiological monitoring solution. In some embodiments, a wireless transceiver (not shown) may also be included in system 300 to enable wireless communication with other components of system 10. For example, pre-processor 320 may output signal 316 (e.g., which may be a pre-processed photoacoustic signal) over BLUETOOTH, IEEE 802.11, WiFi, WiMax, cable, satellite, Infrared, any other suitable transmission scheme, or any combination thereof. In some embodiments, a wireless transmission scheme may be used between any communicating components of system 300.

It will also be understood that while some of the equations referenced herein are continuous functions, the processing equipment may be configured to use digital or discrete forms of the equations in processing the acquired photoacoustic signals.

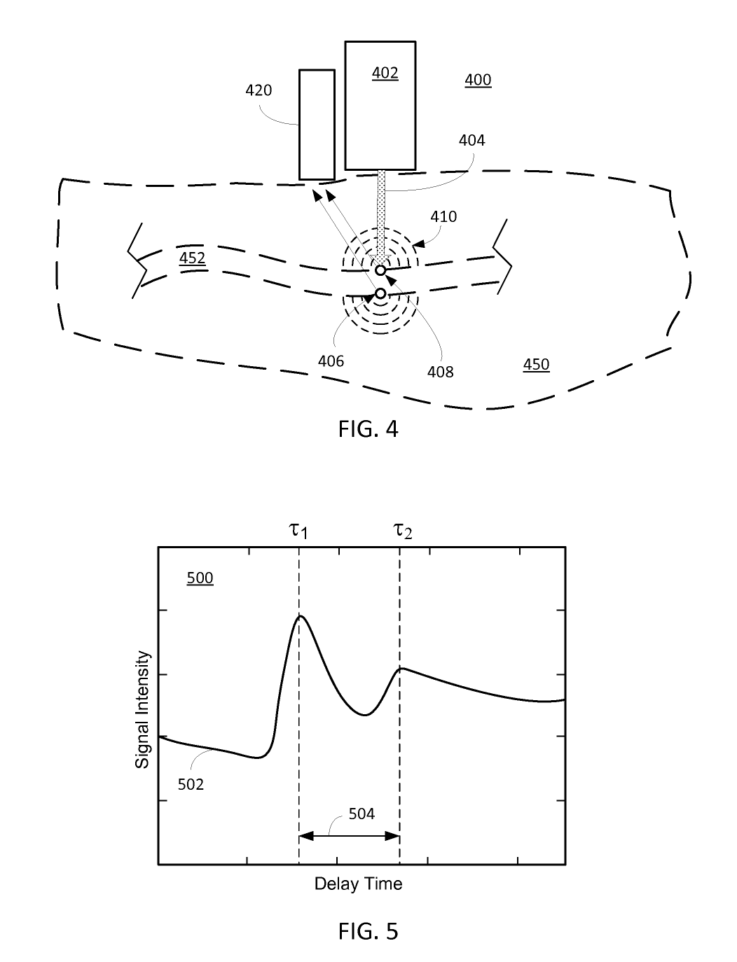

FIG. 4 is an illustrative photoacoustic arrangement 400, in accordance with some embodiments of the present disclosure. Photoacoustic arrangement 400 may include light source 402, controlled by a suitable light drive (e.g., a light drive of system 300 or system 10, although not shown in FIG. 4), may provide photonic signal 404 to subject 450. Photonic signal 404 may be attenuated along its path length in subject 450 prior to reaching blood vessel 452. A constituent of the blood in blood vessel 452 such as, for example, hemoglobin, may absorb at least some of photonic signal 404. Accordingly, the blood may exhibit an acoustic pressure response via the photoacoustic effect, which may act on the boundary of blood vessel 452. Acoustic pressure signals 410 may travel through subject 450, originating substantially from the front boundary 408 and back boundary 406 of blood vessel 452. Acoustic detector 420 (i.e., a photoacoustic detector) may detect acoustic pressure signals 410 traveling through tissue of subject 450, and output (not shown) a photoacoustic signal that may be processed. Because the path length between point 408 and acoustic detector 420 is shorter than the pathlength between point 406 and acoustic detector 420, it may be expected that acoustic pressure signals from point 408 may reach acoustic detector 420 before acoustic pressure signals from point 406. Additionally, in some arrangements, because the path length between point 408 and acoustic detector 420 is shorter than the path length between point 406 and acoustic detector 420, it may be expected that an acoustic pressure signal from point 408 may exhibit a relatively larger peak than an acoustic pressure signal from point 406. Accordingly, acoustic detector 420 may detect two sequential peaks in acoustic pressure signal 410 generated by photonic signal 404 directed at blood vessel 452.

FIG. 5 is a plot 500 of an illustrative photoacoustic signal 502, including two peaks, in accordance with some embodiments of the present disclosure. The abscissa of plot 500 is presented in units proportional to time (e.g., delay time relative to a light pulse), while the ordinate of plot 500 is presented in arbitrary units of signal intensity. At least a portion of photoacoustic signal 502 corresponds to the acoustic pressure response of blood within a blood vessel. Photoacoustic signal 502 exhibits a first peak and a second peak, located at respective times τ₁ and τ₂. The first peak corresponds to the front boundary of the blood vessel, relatively nearer to the acoustic detector. The second peak corresponds to the back boundary of the blood vessel, relatively further from the acoustic detector. It will be understood that the blood vessel diameter may also be determined by measuring other locations of the blood vessel, such as the change in the right to left sides of the blood vessels when suitable acoustic sensors are used. Time difference 504 between τ₁ and τ₂ indicates the relative difference in delay time between acoustic pressure signals from the front and back boundaries. The signal intensity may correspond to the absorption of the particular constituent of the target area. In some embodiments, analysis of the first and second peaks may allow the determination of one or more physiological parameters. When measuring the blood pressure in an artery, the distance between the two peaks will vary over the cardiac cycle and the different distances may correspond to different pressures.

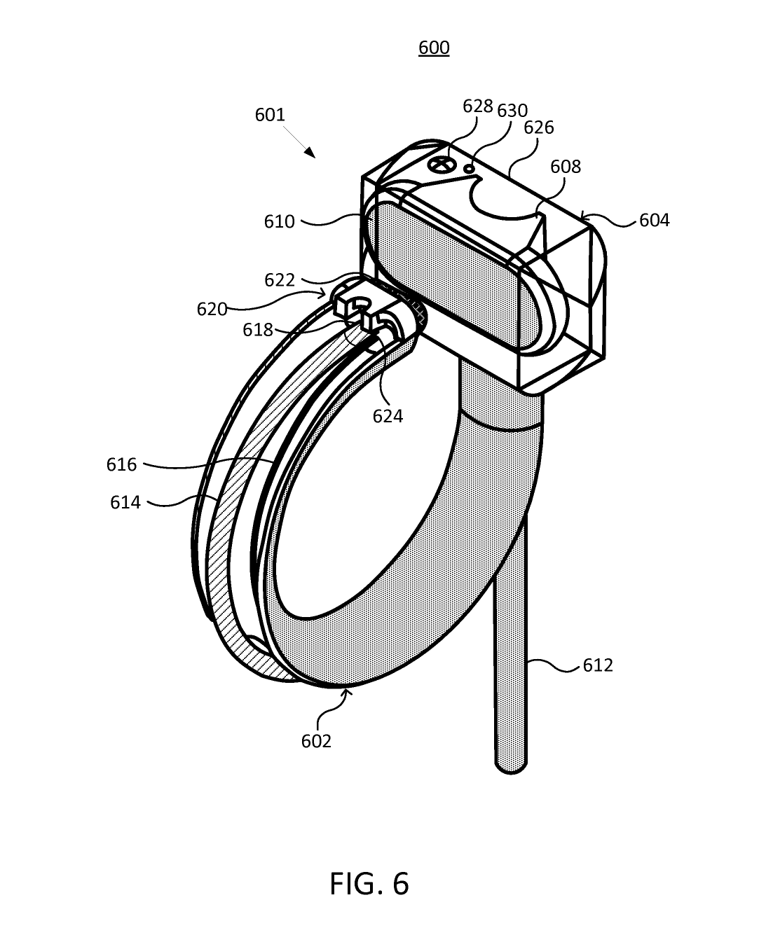

FIG. 6 is an illustrative perspective view of an oral cavity mounted photoacoustic sensing unit 600 in accordance with some embodiments of the present disclosure. Photoacoustic sensing unit 600 may include support assembly 601 and cable 612. Support assembly 601 may include support arm 602, sensor body 620, and cuff 604.

Support assembly 601 may be composed of any suitable material or combination of materials. For example, the material of support assembly 601 may include a biocompatible polymer such as polyvinylchloride (PVC) type polymer, polyethylene (PE) type polymer, polyamide type polymer, other suitable biocompatible polymer, or any combination thereof. The material of support assembly 601 may include biocompatible metal such as stainless steel, titanium, other biocompatible metal, or any combination thereof. The material of support assembly 601 may also include non-biocompatible materials, such as silicon or copper, encased by a biocompatible material such as, but not limited to, those listed above. The material of support assembly 601 may include ceramic materials such as alumina, silica, titanic, other suitable ceramics, or any combination thereof. In addition, the material of support assembly 601 may include any combination of the foregoing materials. In some embodiments, support arm 602 may include low density materials such that the total mass of the oral cavity mounted photoacoustic sensor may be reduced.

In some embodiments, support arm 602 may support sensor unit 620 and cuff 604. Support arm 602 may include a lightweight material as described above and may be shaped in a semi-circular, ellipsoid, or other suitable segment of a toroid such that it may partially surround a target area. The support arm 602, and other elements of support assembly 600, will be understood not to necessarily assume a geometrically perfect toroid or any other shape, but to generally have the characteristics of that shape to the extent that the support assembly can fixably locate sensor unit 620 at the target area.

Support arm 602 may have a hollow cross-sectional shape of a circle, ellipse, oval, other suitable shape, or any combination thereof, such that one or more light pipes 614, one or more signal cables 616, and any other suitable connections (not shown) may be carried through support arm 602. In some embodiments, support arm may have a semi-circular cross-section or other suitable open form, as shown in FIG. 6. In some embodiments, support arm 602 may have a solid cross-sectional form of any shape. Support arm 602 may include clamps, security features, cable strain relief, other suitable features not shown, and any combination thereof to support the elements of sensing unit 600.

In some embodiments, one or more light pipes 614 may carry photons from a light source (not shown) to sensor head 622. In some embodiments, one or more light pipes 614 may be, for example, a 1 mm diameter fiber optic cable, though any suitable size or combination of sizes may be employed. For example, the diameter of the fiber optic cable may between 300 μm and 500 μm, 500 μm and 900 μm, between 900 μm and 1000 μm, or between any other suitable diameter range. One or more light pipes 614 may pass from sensor arm 602 to sensor head 622 at connection point 618. In some embodiments, the light source may be a component of support assembly 601. In some embodiments, the light source may be remote to support assembly 601 and coupled to sensor unit 620 by one or more cables 612, one or more light pipes 614, by any other suitable connection, or any combination thereof. In some embodiments, he light source may be located in sensor head 622 and light pipes 614 may not be used or needed.

In some embodiments, one or more cables 616 may pass from sensor arm 602 to sensor head 622 at connection point 624. One or more cables 616 may couple to sensor head 622 to transmit signals from a photoacoustic detector.

In some embodiments, cuff 604 may be coupled to support arm 602 of support assembly 600. Cuff 604 may include platform 610 to, in part, apply pressure from sensor head 622 to the target area. Bracket 608 may secure platform 610 to support arm 602. Cuff 604 may be of any suitable shape such that a photoacoustic signal is generated and detected by sensor head 622. Cuff 604 may include adjustable element 626 capable of adjusting the pressure generated by platform 610. In some embodiments, adjustable element 626 may be or include an inflatable bladder. Adjustable element 626 may be a bladder that inflates with air, water, gel, other suitable pressurizing medium, or any combination thereof, such that the pressure generated by platform 610 may be appropriately adjusted.

In some embodiments, adjustable element 626 may include a pressure sensor to detect the pressure in the bladder or the pressure applied to sensor head 622. In some embodiments, sensor head 622 may include a pressure sensor to detect the pressure generated by platform 610 and adjustable element 626.

Adjustable element 626 may include pressure relief valve 628 and inflation port 630. Inflation port 630 may function such that air, liquid, any other suitable pressurizing medium, or any combination thereof may be introduced to adjustable element 626. Pressure relief valve 628 may function such that an excess amount of pressurizing medium can be introduced to adjustable element 626 without raising the pressure within adjustable element 626 above a desired pressure. Pressure relief valve 628 may release pressurizing medium from adjustable element 626 at pressures above a fixed pressure, or may release pressures above an adjustable pressure. For example, if adjustable element 626 is an inflatable air bladder with pressure relief valve 628 preset to release pressure above 40 mmHg, air may be introduced to adjustable element 626 until the pressure within adjustable element 626 reaches 40 mmHg; additional air added through inflation port 630 after the pressure within adjustable element 626 has reached 40 mmHg may be released through pressure relief valve 628.

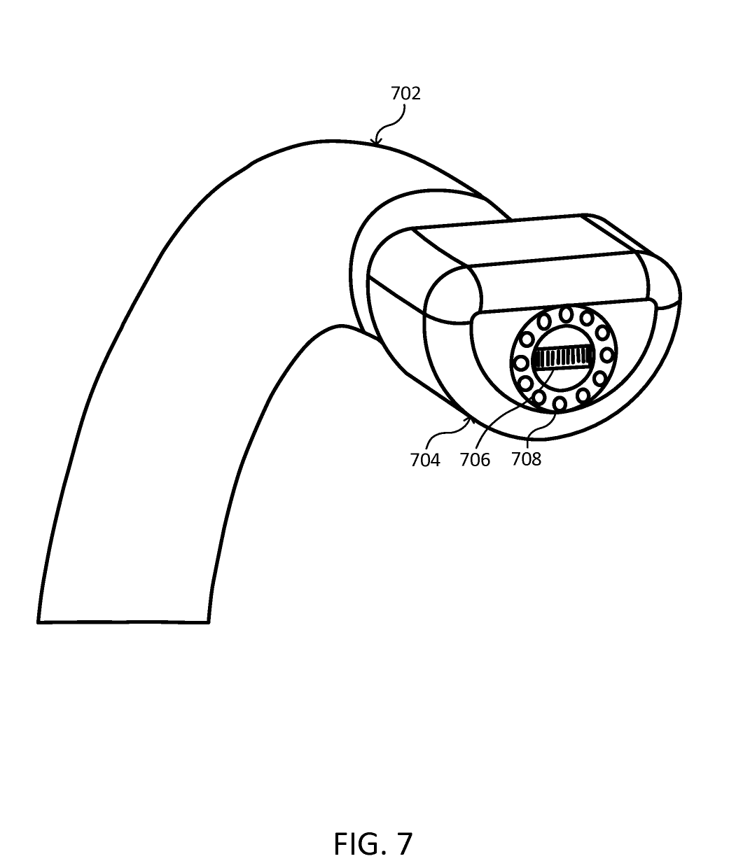

FIG. 7 is a further illustrative perspective view of an oral cavity mounted photoacoustic sensing unit in accordance with some embodiments of the present disclosure. In some embodiments, sensor head 704 of an oral cavity mounted photoacoustic sensor may include one or more light sources 708, one or more photoacoustic detectors 706, and support arm 702.

In some embodiments, support arm 702 couples sensor head 704 to the remainder of the oral cavity mounted photoacoustic sensor units (not shown). Support arm 702 may include a lightweight polymer or other suitable materials as described above. Support arm 702 may be hollow in cross-section and contain cables as described above.

Sensor head 704 may include a plurality of light sources 708 as illustrated in FIG. 7. In some embodiments, the plurality of light sources may share a single fiber optic source, including multiple strands of fiber optic wire carried through support arm 702 and divided into smaller bundles to form the plurality of sources 708. The plurality of light sources 708 as shown in FIG. 7 may also be formed by reflecting, diffracting, diffusing, conducting, or otherwise transmitting light received from elements of support arm 702. In some embodiments, fiber optical cables may be used with a diameter of, for example, 400 μm.

Photoacoustic detector 706 may be an array of linear detectors as shown, a disk detector, a single linear detector, any other suitable photoacoustic detector, or any combination thereof.

Support arm 702 may be flexible or bendable in such a way that sensor head 704 may be adjusted or conform to tissue to improve the photoacoustic measurement. Support arm 702 may be semi-rigid to be custom formed to fit subject's anatomy.

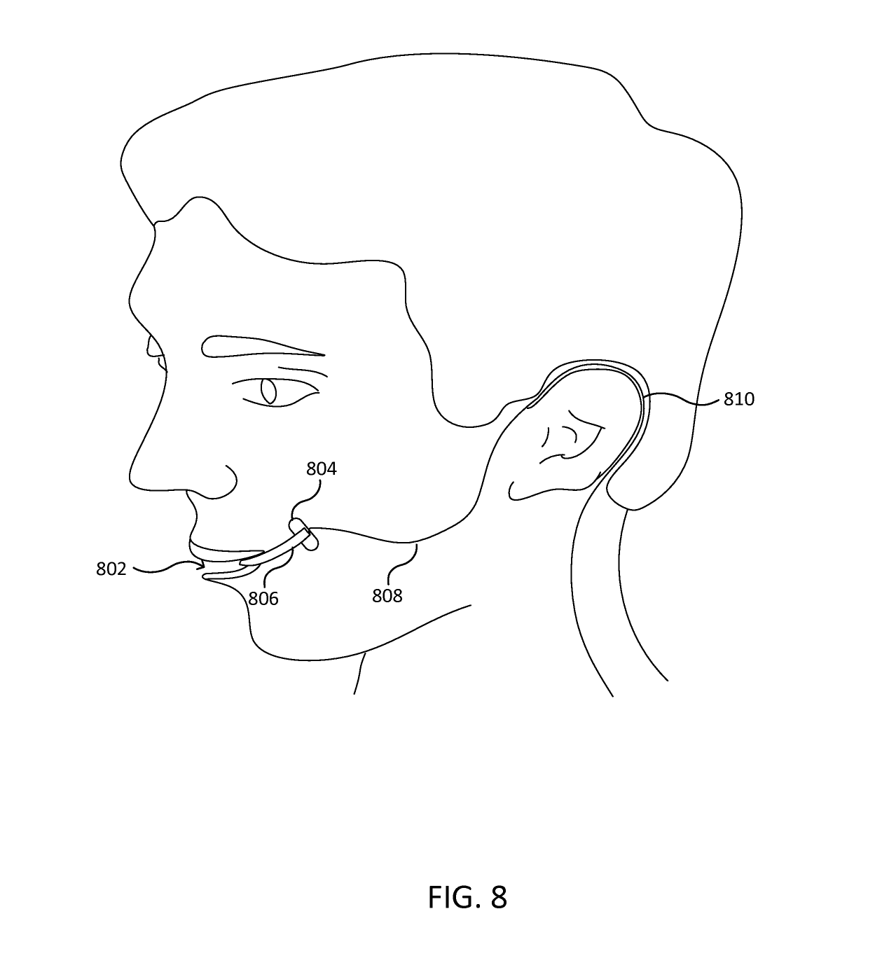

FIG. 8 is an illustrative perspective view of an oral cavity mounted photoacoustic sensing unit mounted to the head of a subject in accordance with some embodiments of the present disclosure. The oral cavity mounted photoacoustic sensing unit may include support arm 806, cuff 804, and wire 808. Support arm 806 may enter the mouth of the subject at 802. The sensor head (not shown) may be located on the buccal tissue substantially near to the relative position of cuff 804 on the inner surface of the subject. For example, the sensor head may be located such that cuff 804 and sensor head (not shown) are separated by the buccal tissue, with the buccal mucosa impinging upon the sensor head. In some embodiments, loop 810 may secure wire 808 to the ear of the subject. Wire 808 may be secured using any suitable contour, adhesive, clip, or any combination thereof.

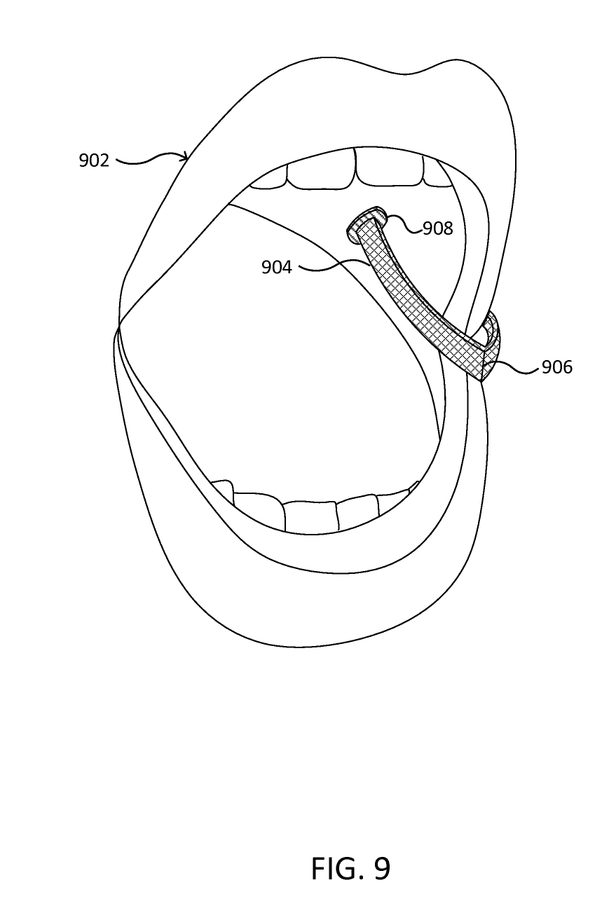

FIG. 9 is an illustrative perspective view of an oral cavity mounted photoacoustic sensing unit impinging on buccal tissue, in accordance with some embodiments of the present disclosure. As illustrated, the mouth of subject 902 is open. Sensor head 908 may impinge upon the buccal mucosa. Support arm 904 may couple the sensor unit to the remainder of the elements of the oral cavity mounted photoacoustic sensing unit (not shown). Bend 906 in support arm 904 may aid in conforming support arm 904 to the subject.

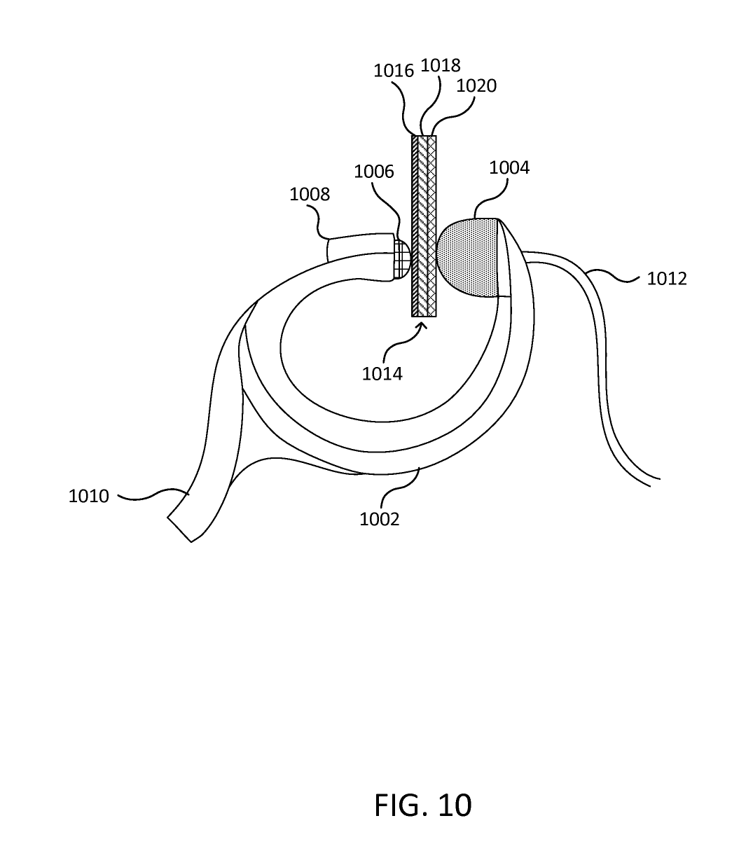

FIG. 10 is an illustrative plan view of an oral cavity mounted photoacoustic sensing unit support assembly in accordance with some embodiments of the present disclosure. In some embodiments, buccal tissue 1014 may be located between sensor head 1006 and cuff 1004. Support arm 1002 may be coupled to sensor head 1006, cuff 1004, cable attachment 1008, cable 1012, and cable routing 1010.

Cuff 1004 may include an inflatable bladder such that adjusting the pressure within the bladder adjusts the pressure applied to the target area of the subject. In some embodiments, tube 1012 may be used to sense pressure in cuff 1004. In some embodiments, tube 1012 may be used to inflate cuff 1004. Cable routing 1010 may be used to support one or more light pipes coupled to sensor head 1006. Cable routing 1010 may have a shape such that the radius of curvature of coupled light pipes may be controlled.

Cable attachment 1008 may be coupled to support arm 1002 and sensor head 1006. Cable attachment 1008 may support optical fibers and other cables as cable and fibers enter sensor head 1006. Cable attachment 1008 may also act as a strain relief.

Buccal tissue 1014 is illustrated as a cross-section, including buccal mucosa 1016, muscle layer 1018, and epidermal skin 1020. In some embodiments, sensor head 1006 may be fixably located proximal to buccal mucosa 1016. In some embodiments, cuff 1004 may impinge upon epidermal skin 1020.

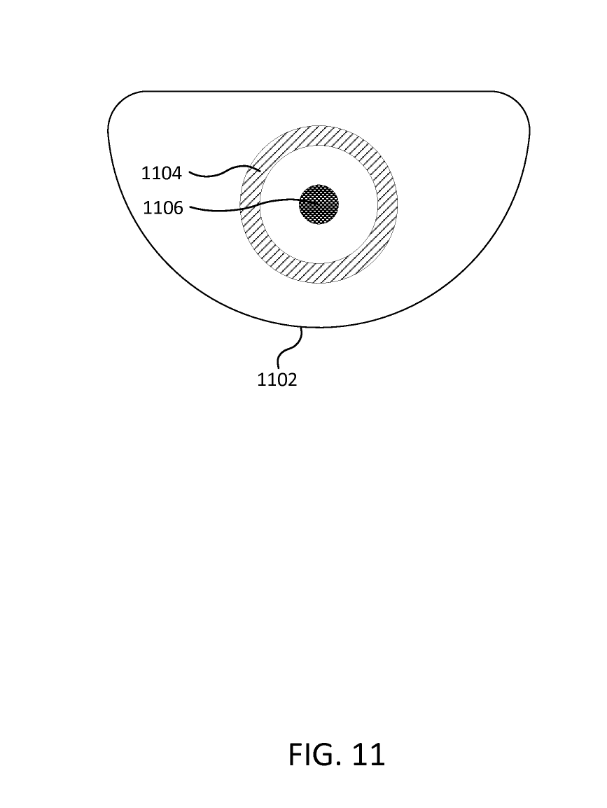

FIG. 11 is an illustrative plan view of an oral cavity mounted photoacoustic sensing head 1102 with an annular photoacoustic detector 1104 and a disk light source 1106 in accordance with some embodiments of the present disclosure. Annular photoacoustic detector 1104 may include one or more ultrasound detectors capable of detecting a photoacoustic signal. Annular photoacoustic detector 1104 may be located on a portion of the surface of sensor head 1102 in a disk or circular shape. For example, as illustrated in FIG. 11, annular photoacoustic detector 1104 may be a detector with a hollow circular shape capable of detecting an ultrasonic signal. In some embodiments, annular photoacoustic detector 1104 may be any suitable shape (e.g., oval, rectangular, etc.) that surrounds disk light source 1106. Disk light source 1106 may include a disk on the surface of sensor head 1102 of any suitable size and shape. In some embodiments, light may be directed to disk light source 1106 by one or more light pipes.

In some embodiments, the photoacoustic detector and the light source of FIG. 11 may be reversed, such that the photoacoustic detector is in the center and the light source surrounds the detector. Light may be directed to the surrounding light source by dividing a bundle of fiber optic cables into individual fibers or smaller bundles of fiber that are arranged around the detector. Light may be directed to the light source by directing fiber optic cables to a plurality of points along an annulus covered with a transparent or translucent diffuser. As used herein, a diffuser will be understood to be a material capable of diffusing, spreading, scattering, or otherwise redirecting light such that there is substantially constant light intensity through the diffuser body. For example, a diffuser may include glass or plastic with a high surface area created by etching or frosting, a translucent polymer, or an internally diffracting material such as opal. In some embodiments, fiber optic cables may terminate at a diffuser ring.

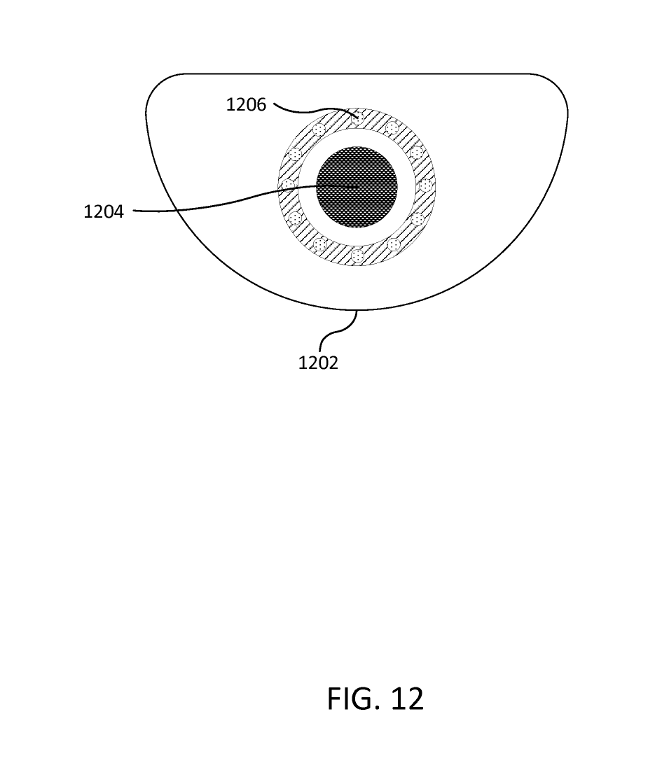

FIG. 12 is an illustrative plan view of an oral cavity mounted photoacoustic sensing head with spot light sources and a disk detector in accordance with some embodiments of the present disclosure. In some embodiments, sensor head 1202 may include light source 1206 and photoacoustic detector 1204.

Light source 1206 may include an annulus located at the surface of sensor head 1202. Light may be directed to light sources 1206 by dividing a bundle of fiber optic cables into individual fibers or smaller bundles of fiber that are arranged in sensor head 1202 in the formation illustrated in FIG. 12. In some embodiments, light source 1206 may be, for example, 400 μm fiber optic cables terminating at prisms on the surface of sensor head 1202. Light source 1206 may include an annulus covered with a transparent or translucent diffuser.

Photoacoustic detector 1204 may be one or more ultrasound detectors capable of detecting a photoacoustic signal. Photoacoustic detector 1206 may be located on a portion of the surface of sensor head 1202 in a disk or circular shape.

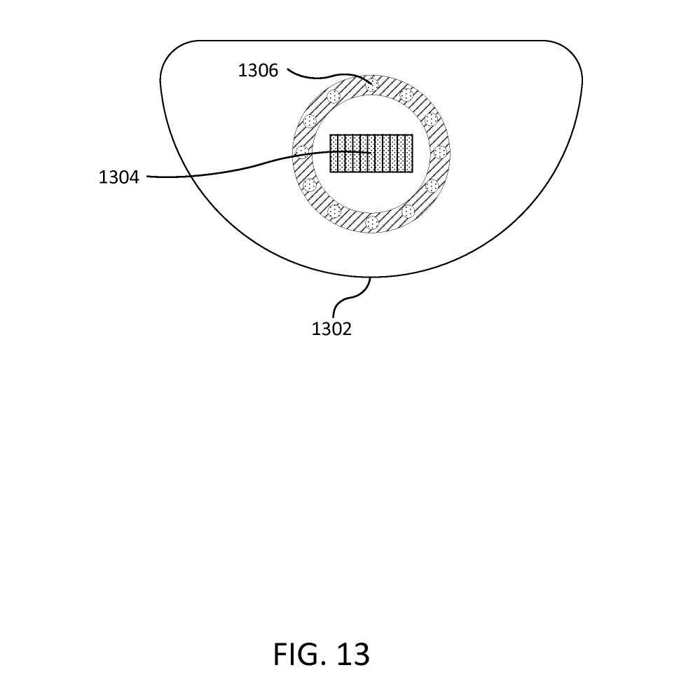

FIG. 13 is an illustrative plan view of an oral cavity mounted photoacoustic sensing head with spot light sources and an array detector in accordance with some embodiments of the present disclosure. In some embodiments, sensor head 1302 may include light source 1304 and photoacoustic detector 1306.

Light source 1306 may be coupled to an annulus located at the surface of sensor head 1302. Light may be directed to light sources 1306 by dividing a bundle of fiber optic cables into individual fibers or smaller bundles of fiber that are arranged in sensor head 1302 in the formation illustrated in FIG. 13. Light sources 1306 may be covered with a diffusing ring as described above.

Photoacoustic detector 1304 may be one or more ultrasound detectors capable of detecting a photoacoustic signal. Photoacoustic detector 1304 may be a linear array of ultrasound detectors. As used herein, an array detector will be understood to be a plurality of adjacent detectors arranged spatially in 1, 2, or 3 dimensions. For example, a linear array detector may include several adjacent detectors arranged evenly in 1 dimension. Photoacoustic detector 1304 may be recessed into sensor head 1302. Photoacoustic detector 1304 may include an ultrasound coupling gel. The ultrasound coupling gel may be impedance matched to the photoacoustic signal. The ultrasound coupling gel may be, for example, LIM6030 ultrasound coupling gel.

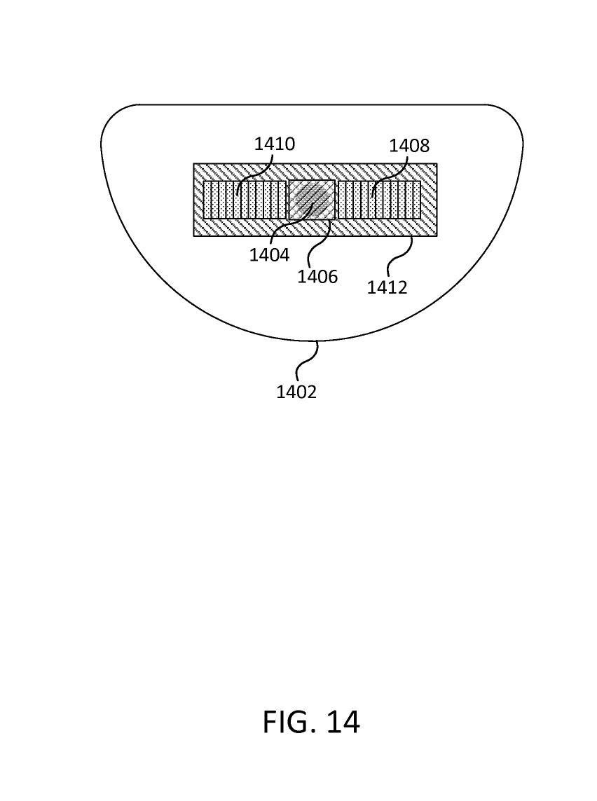

FIG. 14 is an illustrative plan view of an oral cavity mounted photoacoustic sensing head with a diffused spot light source and array detector in accordance with some embodiments of the present disclosure. Sensor head 1402 may include light source 1404, optical element 1406, photoacoustic detector arrays 1408 and 1410, and ultrasound coupling gel 1412.

Light source 1404 may include a round light source on the surface of sensor head 1402. For example, a 1 mm fiber optic cable may reach the surface of or exit sensor unit 1402 at light source 1404. Light may exit light source 1404 through optical element 1406. Optical element 1406 may be a diffuser, mirror, prism, other suitable optical element, or any combination thereof capable of directing light such that the intensity or quality of the photoacoustic signal is modified. In some embodiments, sensor head 1402 may include, for example, a 1 mm fiber optic cable terminating in a square diffuser.

Photoacoustic detectors 1408 and 1410 may be one or more ultrasound detectors capable of detecting a photoacoustic signal. Photoacoustic detectors 1408 and 1410 may each be a multi-element ultrasound detector array. As used herein, a multi-element detector will be understood to be a detector including a plurality of individual detectors. Individual detectors included in a multi-element detector may be similar in design, different in design, or any combination thereof.

Sensor head 1402 may include ultrasound coupling gel 1412. The ultrasound coupling gel may be impedance matched to the photoacoustic signal. Ultrasound coupling gel 1412 may be a gel, hydrogel, hydrocolloid, other suitable material capable of conforming to the target area and transmitting the photoacoustic signal, or any combination thereof. Ultrasound coupling gel 1412 may be, for example, LIM6030 ultrasound coupling gel.

It will be understood that the embodiments of the sensor head design of the oral cavity mounted photoacoustic sensing unit illustrated in FIG. 11, FIG. 12, FIG. 13, and FIG. 14 are provided as illustrative examples of how the light source and photoacoustic detector elements may be arranged. Other suitable arrangements of these elements, and combinations of the elements, may be utilized by the oral cavity mounted photoacoustic sensing unit in accordance with the present disclosure.

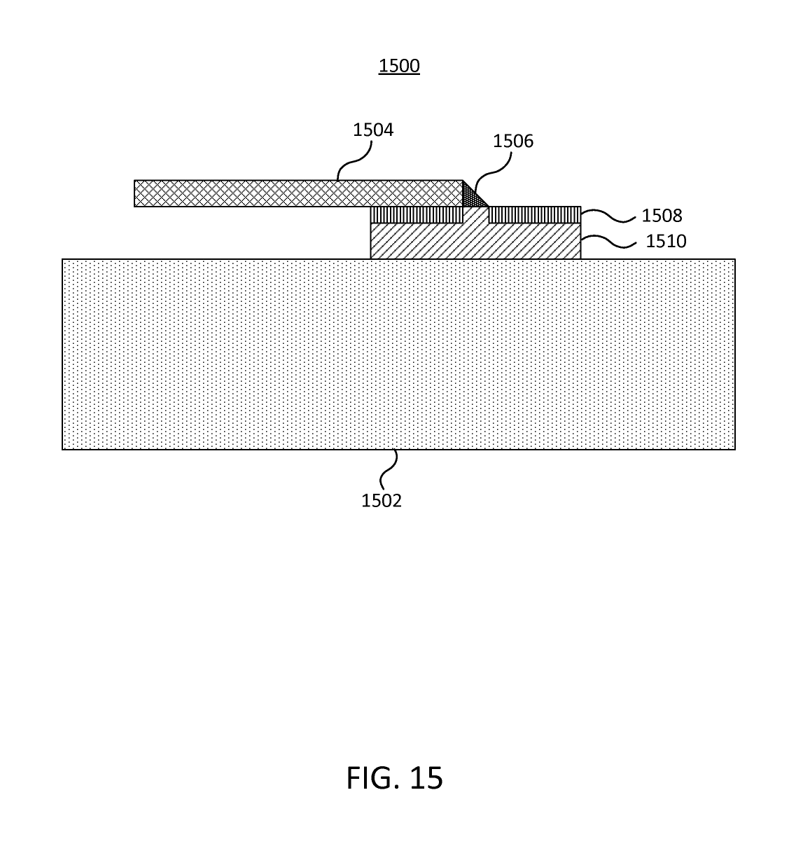

FIG. 15 is an illustrative plan drawing of an optical element that may be employed in an oral cavity mounted photoacoustic sensing unit in accordance with some embodiments of the present disclosure. Optical element 1500 may include light pipe 1504, prism 1506, photoacoustic detector array 1508, and acoustic transport medium 1510. The target area of a subject 1502 may be located in suitable proximity to optical element 1500.

Acoustic transport medium 1510 may be any suitable material that allows for the efficient acoustic coupling and transfer of a photoacoustic response signal from a subject to detector 1508, such as a hydrogel, colloid, hydrocolloid, adhesive, polymer, gel, or liquid. Acoustic transport medium 1510 may displace the air gap between the tissue and the sensor such that acoustic transport medium 1510 may improve coupling of the photoacoustic response in the subject and the photoacoustic detector. Acoustic transport medium 1510 may be impedance matched with the photoacoustic response within the subject, thereby reducing signal degradation and increasing signal power transfer. In some embodiments, a flexible acoustic transport medium 1510 may help mitigate changes in the nature of the photoacoustic detector contact when the subject moves. The acoustic transport medium may have an adhesive applied to one or both sides to further strengthen the interaction with either or both the detector and the subject.

In some embodiments, acoustic transport medium 1510 may be continuously replenished by an appropriate supply mechanism (not shown). Acoustic transport medium 1510 may be replenished from a small reservoir in the sensor body, from a remote reservoir, in any other suitable location, or any combination thereof. Acoustic transport medium 1510 may be carried from the remote reservoir using a tube, pipe, any other suitable carrying equipment, or any combination thereof.

Light pipe 1504 may communicate light from a remote light source to optical element 1500, terminating at prism 1506. In some embodiments, light pipe 1504 may be a glass, polymer, other suitable waveguide fiber, or any combination thereof, capable of transmitting photons. In some embodiments, light pipe 1504 may be replaced with an LED, laser, or other suitable light source.

Prism 1506 may be used to diffuse, divide, guide, lens, or otherwise redirect light from the light source to improve the performance of the oral cavity mounted photoacoustic sensing unit. In some embodiments, prism 1506 may be an optically transparent or translucent optical element made from, for example, glass or polymer. In some embodiments, prism 1506 may include an optical element capable of redirecting light by internal reflection or refraction. In some embodiments, prism 1506 may include a mirror or lens. In embodiment, prism 1506 may include a diffuser capable of defocussing and spreading photons. Prism 1506 may guide light from light pipe 1504 and direct it to a target area of subject 1502. Prism 1506 may increase the amplitude or quality of the photoacoustic signal generated in target area 1502. For example, prism 1506 may increase the amplitude or quality of the photoacoustic signal detected by photoacoustic detector array 1508 by guiding light from the light source toward a specific target area in subject 1502 in proximity to ultrasonic detector 1508.

In some embodiments, one or more optical elements 1500 may be employed at the termination of fiber optic light sources on the surface of a sensor unit. For example, in some of the embodiments illustrated in FIG. 11, optical element 1500 may include a continuous ring capable of diffusing and directing light impinging on the target area of a subject. The signal detected by, for example, photoacoustic detector 1106 may be enhanced or improved by the use of optical element 1500. In a further example as illustrated in some of the embodiments illustrated in FIG. 12, a plurality of optical elements 1500 may be employed at the each of the light sources 1206, in order to direct the light toward the center of the ring and closer to photoacoustic detector 1204.

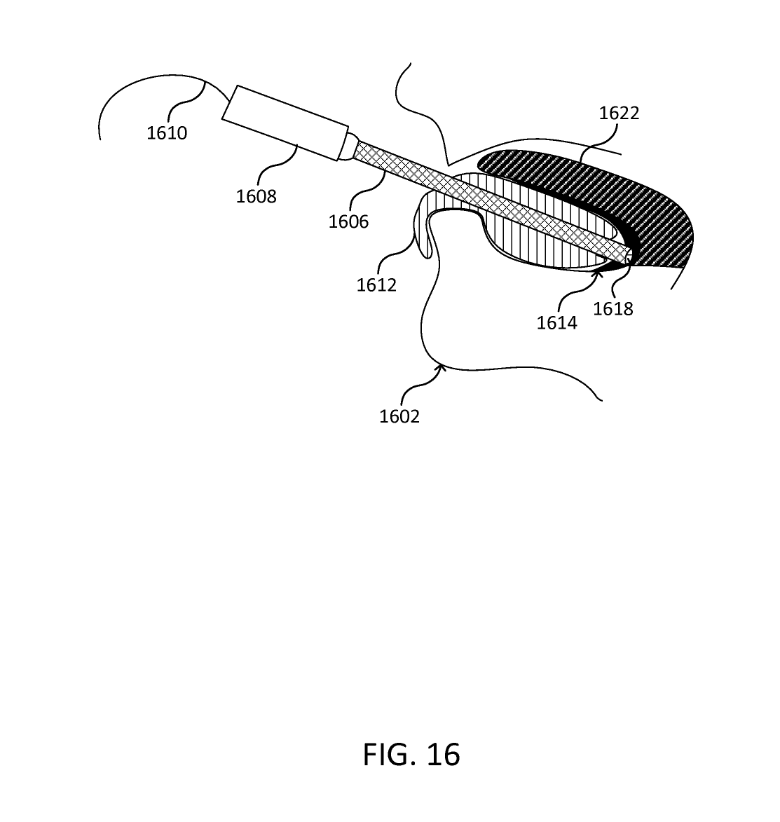

FIG. 16 is an illustrative plan drawing of a sublingual sensing unit in accordance with some embodiments of the present disclosure. A sensing unit may be located such that the target area is sublingual tissue. Photoacoustic sensor head 1618 may be located such that it impinges upon sublingual tissue 1614, under tongue 1622 of subject 1602. Sensor head 1618 may include a light source, detector, other equipment suitable for photoacoustic measurements, or any combination thereof.

Photoacoustic sensor head 1618 may be coupled to sensor support 1606 and handle 1608. Sensor support 1606 and handle 1608 may contain optical, electronic, and other suitable elements for performing photoacoustic measurements. For example, a laser diode light source may be located in handle 1608 and photons generated may be carried by a light pipe through sensor support 1606 to photoacoustic sensor head 1618. The photoacoustic detector included in photoacoustic sensor head 1618 may include an ultrasonic detector which generates an electrical signal. The electrical signal generated by the photoacoustic detector may be carried by a conductive cable from photoacoustic sensor head 1618 along sensor support 1606 to handle 1608. In some embodiments, the arrangement of sensor head 1618 may be similar to or the same as the sensor head arrangements illustrated in FIG. 11, FIG. 12, FIG. 13, and FIG. 14.

Handle 1608 may include one or more light sources (not shown), processing equipment, a power source, other suitable equipment for photoacoustic measurements, or any combination thereof. In some embodiments, handle 1608 may include one or more substantially monochromatic, coherent light sources such as a laser or laser diode. In some embodiments, handle 1608 may include a continuous wavelength xenon light source, mercury discharge, tungsten filament, other suitable continuous wavelength light source, or any combination thereof. In some embodiments, handle 1608 may include one or more light emitting diode light source. Handle 1608 may include processing equipment such as amplifiers, preamplifiers, filters, and other suitable equipment for modifying a signal before transmitting it to a monitor or other device. Handle 1608 may also contain processing equipment to determine a physiological parameter. Handle 1608 may include a display (not shown) to display the physiological parameter.

It will be understood that some or all of the processes described above required for performing a photoacoustic measurement may be carried out in handle 1608, some or all may be carried out in a remote monitor, or any combination thereof. For example, a light source may be contained within handle 1608 while substantially all of the processing steps may be carried out by a remote monitor. In another example, the light source and the processing steps may be implemented remotely. In another example, the light source(s), processing steps, and display are contained within handle 1608 and no remote monitor is employed.

Connection 1610 may include one or more electrically conductive elements, one or more optically conductive elements, or any combination thereof. Electrically conductive elements may include copper, silver, gold, aluminum, carbon, other suitable material capable of transmitting an electrical signal, or any combination thereof. Optically conductive elements may include glass fiber, polymer fiber, other suitable fiber capable of transmitting photons, or any combination thereof. Connection 1610 may include a wireless connection capable of transmitting signals, receiving signals, or both transmitting and receiving signals, via amplitude modulated RF, frequency modulated RF, BLUETOOTH, IEEE 802.11, WiFi, WiMax, cable, satellite, infrared, any other suitable transmission scheme, or any combination thereof.

The sublingual sensing unit may include sensor positioning device 1612. Sensor positioning device 1612 may encircle sensor support 1606 and be shaped such that photoacoustic sensor head 1618 remains fixed near sublingual tissue 1614. In some embodiments, sensor positioning device 1612 may be a solid body comprising a rigid or semi-rigid material such as polyethylene, polystyrene, polypropylene, polydimethylsiloxane, silicone, natural rubber, nylon, neoprene, hydrocolloid, hydrogel, other suitable material, or any combination thereof. In some embodiments, sensor positioning device 1612 may be an adjustable bladder such that its size can be adjusted to fit subject 1602 by filling the bladder with air, liquid, gel, other suitable material, or any combination thereof. The exterior of the bladder may include a rigid or semi-rigid material such as polyethylene, polystyrene, polypropylene, polydimethylsiloxane, silicone, natural rubber, nylon, neoprene, hydrocolloid, hydrogel, other suitable material, or any combination thereof. In some embodiments, sensor positioning device 1612 may include a tab portion that extends out and curves around the lower lip of the subject to aid is fixing photoacoustic sensor head 1618 to sublingual tissue 1614. For example, the tab portion may restrict axial movement of the sublingual sensing unit.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed:

1. An oral cavity mounted photoacoustic sensing unit comprising:
a light source, wherein the light source is configured to transmit light to a subject, and wherein the transmitted light is capable of generating a photoacoustic response in the subject;
a photoacoustic detector configured to detect the photoacoustic response; and
a support assembly configured to secure the photoacoustic sensor unit to the subject and coupled to the light source and photoacoustic detector, wherein the light source and photoacoustic detector are fixably located relative to buccal or sublingual tissue.

2. The oral cavity mounted photoacoustic sensing unit of claim 1, wherein the light is transmitted by at least one light pipe to a target area.

3. The oral cavity mounted photoacoustic sensing unit of claim 2, wherein the light pipe comprises fiber optic fibers with a diameter between 300 μm and 500 μm.

4. The oral cavity mounted photoacoustic sensing unit of claim 2, wherein the light pipe comprises fiber optic fibers with a diameter between 500 μm and 900 μm.

5. The oral cavity mounted photoacoustic sensing unit of claim 2, wherein the light pipe comprises fiber optic fibers with a diameter between 900 μm and 1100 μm.

6. The oral cavity mounted photoacoustic sensing unit of claim 1, wherein the light source comprises one or more laser diodes and wherein the wavelength of the laser diodes is between 600 nm and 1000 nm.

7. The oral cavity mounted photoacoustic sensing unit of claim 1, wherein the light source comprises one or more optical elements to direct light such that the photoacoustic response is enhanced.

8. The oral cavity mounted photoacoustic sensing unit of claim 7, wherein the one or more optical elements comprise a prism.

9. The oral cavity mounted photoacoustic sensing unit of claim 7, wherein the one or more optical elements comprise a diffuser.

10. The oral cavity mounted photoacoustic sensing unit of claim 1, further comprising a plurality of light sources, wherein the plurality of light sources is arranged around the photoacoustic detector.

11. The oral cavity mounted photoacoustic sensing unit of claim 1, wherein the photoacoustic detector comprises a linear piezoelectric ultrasound receiver array of at least one dimension.

12. The oral cavity mounted photoacoustic sensing unit of claim 1, wherein the photoacoustic detector comprises a multi-element detector array.

13. The oral cavity mounted photoacoustic sensing unit of claim 1, wherein the photoacoustic detector comprises a disk detector.

14. The oral cavity mounted photoacoustic sensing unit of claim 1, wherein the photoacoustic detector comprises an annular detector.

15. The oral cavity mounted photoacoustic sensing unit of claim 1, further comprising an acoustic transport medium coupled to the support assembly, wherein the acoustic transport medium is located at least partially between the target area and the photoacoustic detector.

16. The oral cavity mounted photoacoustic sensing unit of claim 15, wherein the acoustic transport medium comprises a hydrogel, colloid, or hydrocolloid.

17. The oral cavity mounted photoacoustic sensing unit of claim 15, wherein the acoustic transport medium is impedance matched to the photoacoustic response of the target area.

18. The oral cavity mounted photoacoustic sensing unit of claim 1, wherein the support assembly further comprises an adjustable cuff.

19. The oral cavity mounted photoacoustic sensing unit of claim 18, wherein the adjustable cuff further comprises a bladder capable of adjusting pressure applied by the cuff to a target area.

20. The oral cavity mounted photoacoustic sensing unit of claim 1, further comprising a pressure sensor capable of sensing a pressure related to the pressure applied to a target area by the photoacoustic sensor.

21. The oral cavity mounted photoacoustic sensing unit of claim 1, wherein the support assembly comprises a bio-compatible solid polymer.

22. The oral cavity mounted photoacoustic sensing unit of claim 1, wherein the support assembly comprises a support arm coupled to the light source and the photoacoustic detector and wherein the support arm has a hollow cross-sectional shape.
